(19) United States
(12) Patent Application Publication
(10) Pub. No.: US 2013/0253302 A1
(43) Pub. Date: Sep. 26, 2013

(54) Electron Tunneling Putative Energy Field Analyzer

(71) Applicants: Bryon K. Eckert, Lanham, MD (US); Bradley N. Eckert, Mesa, AZ (US)

(72) Inventors: Bryon K. Eckert, Lanham, MD (US); Bradley N. Eckert, Mesa, AZ (US)

(73) Assignee: AURAPROBE, INC., Mesa, AZ (US)

(21) Appl. No.: 13/619,821

(22) Filed: Sep. 14, 2012

Related U.S. Application Data
(63) Continuation-in-part of application No. 12/472,174, filed on May 26, 2009, now Pat. No. 8,295,903.
(60) Provisional application No. 61/056,000, filed on May 25, 2008.

(51) Int. Cl. A61B 5/05

(52) U.S. Cl. CPC A61B 5/05
USPC 600/407

(57) ABSTRACT

A device and method of detecting and analyzing a vital field places a thin film tunneling barrier in the path of vital waves in the vital field. The vital waves pass through the high resistivity thin film electrodes into the tunneling barrier and interfere with the electron tunneling process in the tunneling barrier. Control circuitry and a pulse generator drive the device at a known sampling frequency. The interference produces a beat frequency that is output from the tunneling barrier. By adjusting the sample rate by a known amount, a second beat frequency is produced and the beat frequency shift is used to determine the input frequency of the vital waves. The vital waves are very weak and produce frequencies into the terahertz range, so that the input frequency is undersampled by the device. A circuit is designed to maintain a reasonable cost using currently available technology.

### CROSS-REFERENCE TO RELATED APPLICATIONS

This application is a continuation-in-part and claims the benefit of co-pending U.S. patent application Ser. No. 12/472,174 filed May 26, 2009, which claims the benefit of provisional application No. 61/056,000 filed May 25, 2008.

### FIELD OF INVENTION

This invention relates to pattern analysis. This invention relates particularly to methods and devices for detecting and analyzing energy fields emitted by organisms.

### BACKGROUND

All live organisms emit energy fields, referred to herein as vital fields, which are characterized by the organic processes that produce or modify them. There is a significant amount of skepticism surrounding vital fields because no known scientific instruments can directly detect them. The inability to detect, measure, and describe the energy in a vital field is a problem that inhibits human understanding of biological interactions with the environment. Several researchers have proposed that the vital field comprises longitudinal electromagnetic waves. These are periodic variations in time density somewhat like sound waves in air, extending into the millimeter wave range. The wavelength and time-varying component of the vital waves make them very difficult to detect and analyze.

It is theorized that the presence of the vital field may be indirectly detected using gas discharge visualization (“GDV”) equipment, such as that described by K. Korotkov in 2005. GDV equipment is capable of visualizing a discharge of photons and electrons from a sample that is subjected to a pulsed electromagnetic field. Specifically, a low-current discharge emanating from the sample may be photographed, appearing as a glow or corona around the organism. The corona appears for both organic and inorganic samples. However, while there is no significant variation in the corona of inorganic materials, for organic materials the corona varies greatly. Korotkov proposes that the variation is due to the presence of vital fields, and has shown that in humans the variations may be affected by the subject's medical condition and applied medical treatments. The effect has been shown in over 10,000 clinical trials. However, GDV is not widely accepted as a means for detecting and analyzing vital fields because the interaction of the vital field with the corona discharge is not well understood and the GDV equipment is very expensive and cumbersome.

Most vital field detection devices to date have been either a variation of GDV equipment, which is itself a variation of Kirlian high-voltage photography equipment, or low voltage electric field sensors. One device, used to detect pathogens in an organism, places the organism in an electrical field and detects an aura signature of pathogens energized by the field. Another device uses a passive detector that characterizes pulses of charge transfer called charge density pulses through conductive plates placed near the palms of the hands. The decay envelope of the detected pulse train may provide information useful for analysis of the body's chakra regions. However, the data is extracted from a pulse train that does not achieve a steady state, and so the data that can be obtained is limited.

Some detectors, such as electrocardiographs and electroencephalographs, analyze alternating current waveforms detected by electrodes placed on the skin of the test subject. One known device uses contacts on the palms and fingers to detect the physiological signals of the human body supposedly associated with auras. Other detectors introduce an electric current into the electrodes, such as with a galvanic skin response and others, which measure the organism's interaction with the introduced current through physical contact between the organism and the detector. Still other devices use capacitance to measure the interaction, but must be placed extremely close to the organism to be effective. Contact and capacitance based devices suffer significant problems with artifacts caused by the proximity.

One device capable of detecting the static magnetic component of a wave is the Superconducting Quantum Interference device, or “SQUID.” SQUIDS are highly sensitive, extremely expensive magnetometers. However, SQUIDS only detect the presence of strong waves. A typical vital field generated by an organism has weak vital waves that SQUIDS cannot detect. Further, SQUIDS do not detect the spectral information needed analyze a vital field. A reliable, understandable, and useful detection device and method is needed. Therefore, it is an object of the present invention to reliably detect and analyze vital fields. It is a further object that the vital fields be detected with a device that is relatively inexpensive compared to known devices. It is another object of the invention that the device and method of detection reduces unwanted artifacts by not contacting the organism.

Within the domain of quantum mechanics, the quantum tunneling effect was first observed and applied shortly after the 1926 publication of Erwin Schrödinger's equations describing the wave function of matter in a system. Quantum tunneling occurs because the particle behavior, as modeled by the Schrödinger wave function for a particle traveling in a medium, cannot be discontinuous at the boundary of the medium. Thus, it has been shown that at the boundary, which may be a physical barrier or a barrier formed by a difference in electrical potential, the wave is split into a reflected component and an evanescent component, a phenomenon known as evanescent wave coupling. The reflected wave reflects off of the barrier with a higher energy than the wave had when it encountered the barrier. The evanescent wave propagates, or tunnels, through the barrier and then exhibits exponential decay over distance traveled. In this manner, electrons, photons, and other particles cross a barrier even though they may not have the energy needed to do so. The inventive device utilizes the principles of quantum tunneling to detect and analyze the vital fields as described below.

### SUMMARY OF THE INVENTION

The present device is placed in a vital field such that the vital waves in the vital field are conducted into a detector having a Schottky diode and an electrical pulse generator. The Schottky diode includes a tunneling barrier and two or more electrodes having high sheet resistivity. The Schottky diode is forward DC biased with a current source, and receives a pulsed alternating current from the pulse generator, which may be a step recovery diode or a bipolar junction transistor, preferably operated in its avalanche breakdown region. The vital waves impinge the Schottky diode without electrical contact, traveling through the electrodes of high sheet resistivity and into the tunnel barrier. The vital waves are preferably conducted into the active region of the Schottky diode through a focusing horn.

Control circuitry provides a first control signal at a first sampling frequency to the detector. The first control signal is chosen to undersample the vital waves from the vital field, which may have very high frequency. The first control signal modulates the tunnel current, which is the portion of the current that passes through the tunneling barrier. The first control signal instructs the pulse generator to apply a narrow current pulse to the Schottky diode. The pulsing current “pumps” the Schottky diode, similar to a sampling mixer or sampling phase detector, to create a period of peak tunnel current. During the period of peak tunnel current, the vital waves from the vital field cause a detectable interference with the tunnel current, producing a first mixed signal including a first beat frequency that is the difference between the frequency of the vital waves and a high harmonic of the first sampling frequency.

The first mixed signal is conducted to signal processing circuitry, which filters the signal and applies Fourier transforms. Extraction of the beat frequency from the first mixed signal indicates that the vital waves are present. Then, the control circuitry is adjusted to produce a second control signal and the detection process repeats, producing a second mixed signal with a second beat frequency. The signal processing circuitry uses the first and second beat frequencies to determine the frequency of the vital waves from the vital field. The results of the signal processing are then displayed on a screen. Both the control circuitry and the signal processing circuitry include components that work to limit noise and other artifacts generated during the detection process.

Through continued use of the device, a reference database is developed to associate vital fields with the organisms, organs, organic material, metaphysical changes, or conditions presumed to generate the vital fields.

### BRIEF DESCRIPTION OF THE DRAWINGS

FIG. 1 is a schematic diagram of the present device.

FIG. 2 is a cross-sectional side view diagram of the preferred Schottky diode.

FIG. 3 is a circuit diagram of the preferred embodiment of the present device.

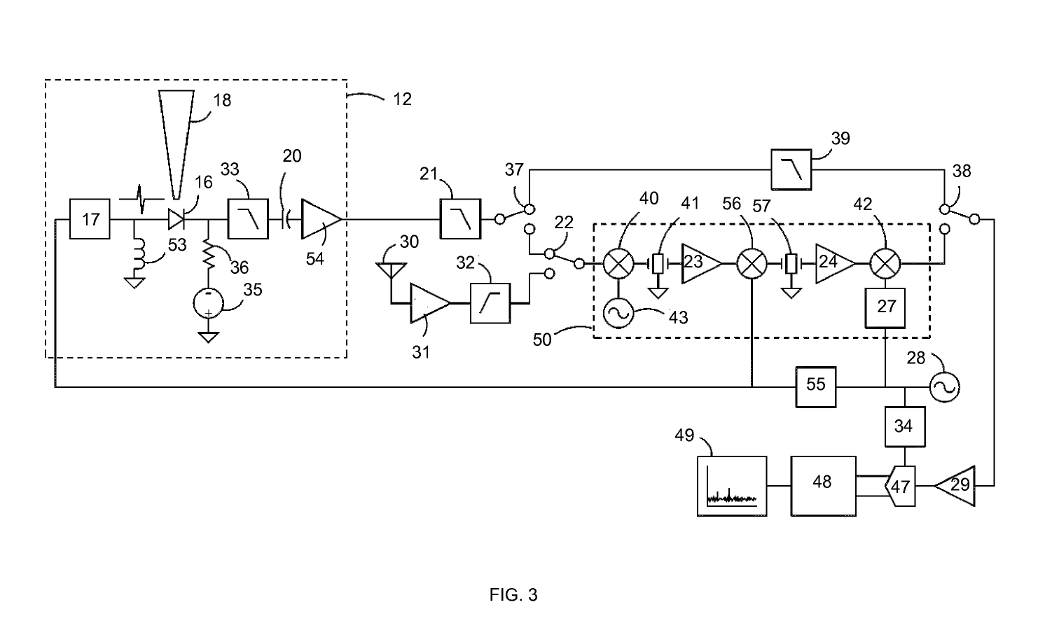

### DETAILED DESCRIPTION OF THE INVENTION

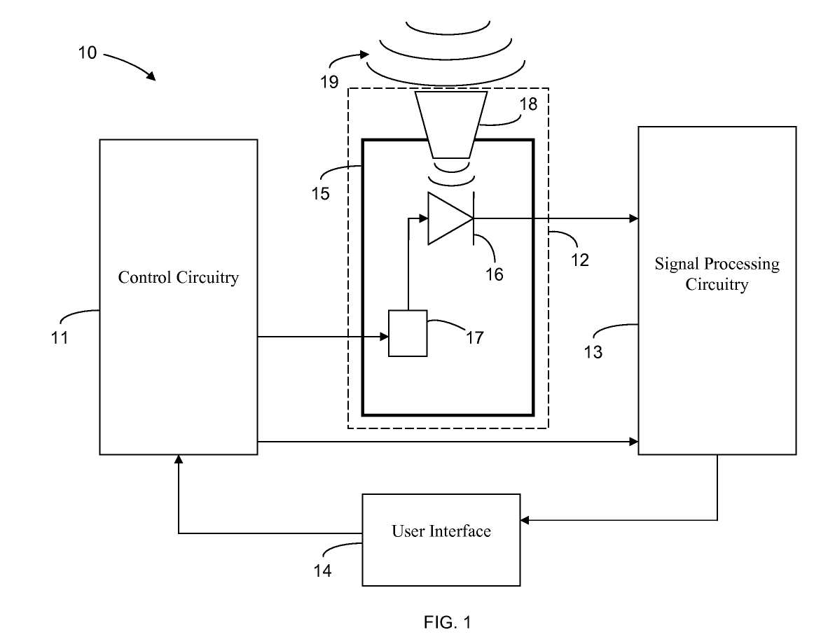

FIG. 1 illustrates the present invention, which is a device 10 for detecting and analyzing vital fields. The device 10 is placed in the path of vital waves 19 that are present in the vital field to be detected. The detection process is initiated through a user interface 14, such as by pressing a button or a designated part of a touch screen that indicates to control circuitry 11 that the process should begin. The control circuitry 11 then generates, as described in detail below, a first control signal having a first sampling frequency, and sends the first control signal to a detector 12. The control circuitry 11 may also send the first control signal to signal processing circuitry 13 for use in a frequency converter as described below.

To achieve the desirably high sensitivity of the device 10, the detector 12 may be substantially enclosed by electromagnetic shielding 15. The shielding 15 protects the internal components of the detector 12 from unwanted interference by light and other electromagnetic waves. The shielding 15 may be a Faraday cage or other shielding structure. The shielding 15 may be a mesh of conducting material, but preferably the shielding 15 is substantially solid and fully encloses the detector's 12 internal components, except that a small opening may be left in the shielding to allow the vital waves 19 to pass into the detector 12. This opening may be covered by an opaque dielectric material (not shown) that blocks light but allows the vital waves 19 to pass. The dielectric material may be any electrical insulator, including insulating tape such as vinyl, plastic, or polyester tape. Preferably, however, the opening is not covered to avoid interference of the covering with the vital waves.

The internal components of the detector 12 may include a Schottky diode 16 configured to facilitate quantum tunneling and an alternating current pulse generator 17 that cooperate to control the parameters of a tunnel current in the diode 16. The diode 16 is forward biased and receives a constant direct current from a current source, producing an average tunnel current across a dielectric barrier in the diode 16. The average tunnel current is preferably between about 3 mA and about 30 mA, most preferably about 15 mA. The pulse generator 17 is electrically connected to the diode 16 to provide a pulsed alternating current to the diode 16. The pulsed current augments the constant current to drive a peak tunnel current in which the presence of vital waves 19 is detectable. The detector 12 may further comprise a focusing horn 18 through which the vital waves 19 pass and are directed into the tunneling region of the diode 16. The focusing horn 18 is made of a conductive material, preferably metal, that will reflect the vital waves 19 due to their static electric component. Suitable metals include brass, copper, and aluminum, but most preferably the focusing horn 18 is brass.

The control circuitry 11 directs the first control signal to the pulse generator 17, causing the pulse generator 17 to apply narrow current pulses to the diode 16 at the first sampling frequency. Preferably, the pulse width is about 50 picoseconds. Suitable pulse generators to achieve the pulsing parameters include a step recovery diode and a bipolar transistor biased in its avalanche region. The current pulse profile may be that of a sine wave. The current pulses augment the constant direct current to generate the peak tunnel current. The tunnel current propagates so quickly, typically within about 50 picoseconds, that the first sampling frequency is retained in the resulting signal that is emitted from the Schottky diode 16. The resulting signal is called the first mixed signal, as described below.

One or more components of the incident vital waves 19, including electrostatic, temporal, and scalar electromagnetic vector components, interfere with the tunneling process in the tunneling region and hence perturb the tunnel current. In the case where the vital waves 19 have extremely high frequencies, of at least 30 gigahertz and further into the terahertz range, undersampling may be used to determine the frequency. The signal propagated through the Schottky diode 16, being the product of oscillating current pulses and the non-linear capacitance versus voltage characteristic and non-linear tunnel current characteristic of the diode 16, has sufficient harmonic content that heterodyning occurs between the vital waves 19 and a high harmonic of the first sampling frequency. As a result, the first mixed signal carried out of the Schottky diode 16 contains a first beat frequency that is the difference between the frequency of the vital waves 19 and a high harmonic of the first sampling frequency.

The first mixed signal is then processed by signal processing circuitry 13. As described below, the first mixed signal undergoes filtration, optional frequency conversion, and Fourier transformation to extract the desired frequency data. During or after this processing, the control circuitry generates a second control signal having a second sampling frequency and sends it to the detector 12, resulting in a second mixed signal having a second beat frequency. The second mixed signal is also processed by signal processing circuitry 13. The second beat frequency is subtracted from the first beat frequency to obtain the beat frequency shift.

The harmonic with which the vital waves 19 were heterodyned is determined by dividing the sampling frequency shift by the beat frequency shift. The harmonic number of the first sampling frequency then allows calculation of the observed frequency imparted by the vital waves 19. The detection process may be repeated with additional sampling frequencies to reduce uncertainties if multiple vital wave 19 frequencies are present. As part of the repeated detection process, the direct current may be removed from the diode 16 to determine if a detected frequency was caused by a vital wave 19 or by electromagnetic noise, such as a radio frequency, affecting the components. That is, if the direct current is removed and the signal disappears from the spectrum analysis, it is more likely that the signal was caused by a vital wave 19 than by unwanted interference.

The spectral data of the detection process may be formatted and displayed on a screen in the user interface 14. Further, the spectral data may be compared to records in a reference database to determine if it matches information gathered on known vital fields. In this manner, if it has been determined that certain data previously gathered by the device 10 correlates to, for example, the presence of a blood disease or its precursors, the results of the detection process may be compared to the previously collected data to determine if the scanned person has the same disease or its precursors. Reference databases may be generated for specific plants and animals, and may be used to detect vital fields associated with bodily states and conditions, the presence or absence of diseases, and aspects of other body energies such as chakra or qi. The reference databases may be stored on the device 10, on a computer to which the device 10 may be electrically connected such as via Universal Serial Bus cable or wireless Ethernet connection, or on a server accessible over the internet, such as in a cloud storage framework.

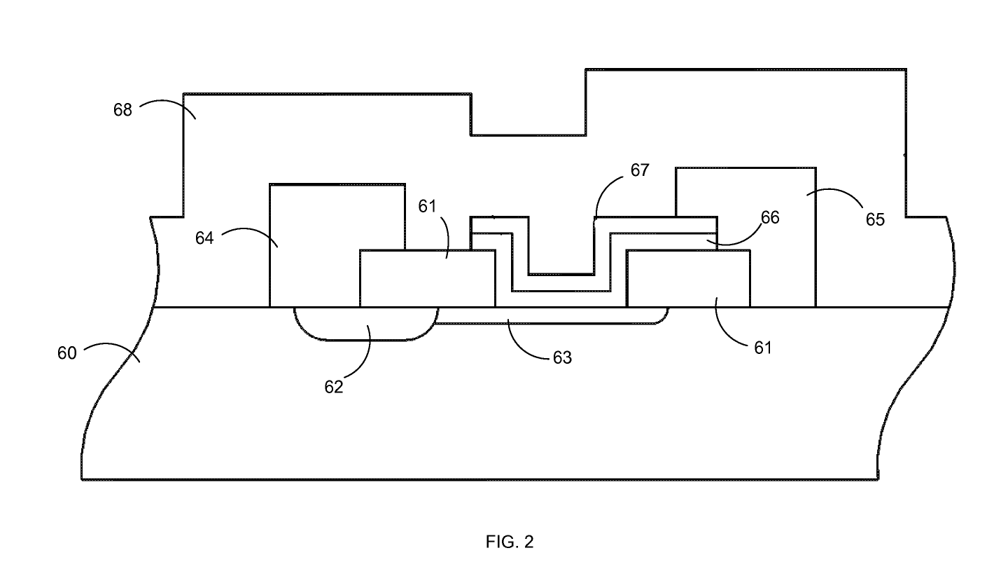

FIG. 2 is a diagram of the preferred Schottky diode 16. The design of the diode 16 may conform to any parameters that facilitate a tunnel current as described below while allowing the vital waves 19 to reach the tunnel current without themselves being disrupted. The diode 16 may use any suitable semiconducting material, including silicon, doped or undoped GaAs, GaN, or another compound semiconductor, as a semi-insulating substrate 61. Preferably, the substrate 60 is high resistivity float zone silicon. The substrate 60 may be refined to atomic flatness to maximize the effectiveness and reliability of the device 10. Chemical etching or mechanical polishing processes may be used to achieve atomic flatness of the substrate 60, depending on the material used. The aspect ratio of the device 10 is kept very high in order to minimize the intrinsic resistance of the device. Preferably, the substrate 60 measures about 200 μm wide by about 1 μm long. These dimensions may approach the limits of proper alignment capabilities of the fabrication device, which is preferably a lithographic device. In that case, the current transmission may be protected by shallow- or deep-trench isolation. The diode 16 may include one or more oxide trenches 61 positioned to reduce the variability of the diode's 16 active area to a single critical dimension, thus increasing the fabrication repeatability of the device. The oxide trench 61 may be made of any dielectric material, and is preferably silicon dioxide applied by plasma-enhanced chemical vapor deposition to avoid thermal damage to the doping regions of the substrate 60 or other components deposited thereon.

The diode 16 is fabricated with electrodes of similarly high sheet resistivity, which will allow the vital waves 19 to partially or fully pass through both electrodes and impinge upon the tunneling region of the diode 16. A heavily-doped region 62 of the substrate 60 creates an ohmic contact to a first bonding pad 64. The first bonding pad 64 is a metal contact point to which bonding wires are connected, and is preferably aluminum if the substrate 60 is silicon or gold if the substrate 60 is gallium arsenide or another compound semiconductor. The heavily doped region 62 and the first bonding pad 64 are configured so they do not prevent the vital waves 19 from reaching the tunneling region of the diode 16 as described below. The base electrode 63 of the diode 16 is a lightly n-doped region of the substrate 60 that abuts the heavily doped region 62 so that electrical conductivity between the first bonding pad 64, heavily doped region 62, and base electrode 63 is maintained. The light doping of the base electrode 63 creates a high bulk resistivity, which permits a Schottky barrier to be formed in the diode 16 and ensures a highly nonlinear capacitance-voltage characteristic. Further, the light doping maintains a high sheet resistivity of the base electrode 63 to allow the vital waves to penetrate into the tunneling region. The base electrode 63 is preferably formed by ion implantation, but may be formed by thermal diffusion or molecular beam epitaxy. If the base electrode 63 is formed by ion implantation, the oxide trenches 61 will insulate the tunnel dielectric 66 and resistive thin film 67 from the edges of the base electrode 63 where the doping may be non-uniform.

The doping profiles for the heavily-doped region 62 and base electrode 63 may depend on the choice of material for the substrate 60 and the degree of electron mobility desired. Preferably, the regions are n-doped in order to provide higher electron mobility than with p-doped regions. In the preferred embodiment, the region of the silicon substrate 60 to comprise the base electrode 63 is doped with phosphorus at a nominal energy of about 100 keV and a dose of about 1×10̂13 ions per cm̂2 to impart the desired characteristics. Alternatively, the substrate 60 is doped with arsenic or, if a p-doping profile is desired, with boron. In another alternative embodiment, where the substrate 60 is undoped gallium arsenide, the base electrode 63 may be formed by molecular beam epitaxy of silicon or tin to create an n-doped profile. Also preferably, the heavily-doped region 62 is doped with phosphorus at a dose of about 1×10̂21 ions per cm̂2 in order to create the ohmic contact with the first bonding pad 64. The dose is applied by thermal diffusion of a spin-on dopant such as Emulsitone Phosphorosilicafilm.

The tunneling region of the diode 16 contains a tunnel barrier 66 and a thin film 67. The width of the tunneling region, which is preferably the distance between oxide trenches 61, is preferably about 1 μm to minimize the intrinsic resistance of the diode 16. The tunnel barrier 66 is a thinly deposited dielectric material, preferably aluminum nitride (AlN), and most preferably at thickness of 1.6 nm. Nitride dielectrics, including silicon nitride as well as aluminum nitride, gallium nitride, and others, are preferred due to their lack of a soft electrical breakdown characteristic, which offers improved reliability of tunnel current over dielectric materials that do have this characteristic. Preferably, the tunnel barrier 66 is formed by Plasma Enhanced Atomic Layer Deposition (“PEALD”) in order to ensure uniform thickness at nano-scale, and further to reduce the costs related to thermal processing of the diode 16. The very thin tunnel barrier 66 allows the capacitance of the diode 16 to change significantly in response to applied voltage, which aids in generating a peak tunnel current. The thin film 67 serves as the top electrode and therefore is metal or another conductor. Preferably, the thin film 67 is a 1 nm layer of titanium nitride (TiN) that is most preferably deposited using PEALD, but may be applied using any known method of thin film deposition. Titanium nitride is preferred due to its wide availability, but a suitable replacement having high sheet resistivity, such as tantalum nitride, may be used.

A second bonding pad 65 makes electrical contact with the thin film 67. The second bonding pad 65 is a metal contact point to which bonding wires are connected, and is preferably aluminum if the substrate 60 is silicon or gold if the substrate 60 is gallium arsenide or another compound semiconductor. Device passivation is realized with a layer 68 of polymeric or other passivating material. Preferably, the passivating layer 68 is made of Parylene about 10 microns thick at its thickest point. Other acceptable materials for the layer 68 include silicon nitride and silicon dioxide. The layer 68 also covers the associated bond wires. Alternatively, the bond wires may be protected by selectively cured UV epoxy, with any uncured epoxy being removed from the active region so that it does not block the vital waves.

The described diode 16 serves as a tunneling structure with very high sensitivity due to the atomic flatness of the components and the nano-scale thickness of the dielectric and thin-film electrode. When a current is applied as described below, in absences of external stimulus, the diode 16 will produce normally-distributed white noise in the form of a decaying tunnel current that travels from the negatively-charged base electron 63 through the tunnel barrier 66 to the thin film 67. The noise power varies proportionately with the amount of current applied. The shot noise of the diode 16 will have a 1/f component that dominates the signal at frequencies below about 100 kHz. The diode 16 design allows vital waves 19, which are scalar electromagnetic waves, to travel through the doped and metallized regions into the tunneling region. A periodic variation in time rate density of the vital waves 19 manifests as a slight variation in tunnel current, and subsequently as a disruption in the normally-distributed white noise generated by the diode 16. The 1/f component may contain the signal contributed by the vital waves and is therefore analyzed as part of the mixed signal described below.

In another embodiment, the tunneling region may be manufactured as a separate component from the Schottky diode 16. In this embodiment, the tunneling region essentially becomes a tunneling component comprising bottom and top thin-film electrodes separated by a tunneling barrier. The dimensions of the tunneling component are the same as those described for the tunneling region, in order to maintain a high sheet resistivity of the electrodes and the sensitivity of the overall device, imparted by the nano-scale thickness and atomic flatness of the dielectric and electrodes. Separating the tunneling component from the Schottky diode 16 allows the use of non-semiconducting materials, such as quartz, sapphire, or another crystalline material, for the substrate 60 because there is no need to dope regions of the substrate 60 for creating a Schottky contact within the tunneling component. The electrodes of the tunneling component are preferably Titanium Nitride.

With the tunneling component separate from the Schottky diode 16, the diode 16 may be an optional component. Specifically, the tunneling component may be electrically connected to the pulse generator 17, which may be configured to apply a pulsed direct current that peaks in one direction, rather than alternating and causing bidirectional tunneling current across the tunneling component. In this configuration, the Schottky diode 16 would not be needed. Alternatively, the Schottky diode 16 may be positioned in the circuit between the pulse generator 17 and the tunneling component. In this configuration, the Schottky diode 16 prevents bidirectional current flow across the tunneling component, so the pulse generator 17 may provide alternating current as described above. By building a separate tunneling component, an off-the-shelf Schottky diode 16 may be used in the circuit. Furthermore, a wider variety of materials may be used in the tunneling component, and in the detector 12 as a whole, because the design is not limited by the requirements of a Schottky contact. The disadvantage of this embodiment over an integrated diode 16 design is that there are more wire contacts and therefore additional inductance in the circuit.

Even with a very high tunnel current density, the overall tunnel current through the tunnel barrier 66 will be small. The small geometry is necessitated by the large capacitance created by the thin tunnel barrier 66. The shot noise current will be reduced at higher intermediate frequencies because this noise, and any corresponding received signal, will be directly across the diode 16 capacitance. Therefore, the preferred embodiment of the present device endeavors to minimize noise in the circuit using components that filter unwanted signals and maintain low impedance on sensitive elements. In particular, the tunneling region aspect ratio is made as wide as possible to minimize the intrinsic resistance of the device 10, which will add directly to the thermal noise output from the diode 16. It will be understood that certain desirable features of the diode 16 are affected in inverse proportion according to the capacitance and intrinsic resistance of the diode 16. In particular, the rectifying effect of the Schottky barrier must be balanced against the overall series resistance of the diode 16; the former affects the efficiency of the harmonic signal mixing, while the latter affects the thermal noise produced by the device. For a given current density, the constant current and capacitance will scale linearly with the size of the tunneling region, and the intrinsic resistance will be a function of the size and aspect ratio of the tunneling region. This combination can vary with the design of the low noise amplifier that is connected to the diode 16 as described below. For example, a typical low cost silicon germanium transistor will have a low impedance noise match and be less sensitive to a relatively high capacitance, generated by the diode 16, on its input. Preferably, the resistivity of the base electrode 63 and thin film 67 is between about 1000 ohms/cm̂2 and 10,000 ohms/cm̂2, and most preferably about 2000 ohms/cm̂2, to achieve the optimum balance between Schottky barrier effect and series resistance

When the detection process is initiated, a signal source generates the first control signal. Preferably, a master clock oscillator 28 supplies the master clock frequency to a frequency multiplier 55, which uses the master clock frequency to produce the first control signal at the first sampling frequency. The master clock oscillator 28 preferably contains one or more digitally tuned crystal oscillators, most preferably having two crystals at frequencies of 45.2 and 46.173 MHz, respectively. Alternatively, the master clock oscillator 28 may be a frequency synthesizer or a voltage controlled crystal oscillator controlled by a digital-to-analog converter. Preferably, the frequency multiplier 55 multiplies the master clock oscillator's 28 first control signal by 22 to 994.4 MHz. The frequency multiplier 55 sends the first control signal into the detector 12 and to the second intermediate frequency (“IF”) mixer 56.

Within the detector 12, the Schottky diode 16 receives the direct current from a current source 35. The voltage level is controlled by an external computer processor. The voltage is adjusted to give a fixed current through a bias resistor 36, producing an average tunnel current within the Schottky diode 16. The pulse generator 17 receives the first control signal from the frequency multiplier 55 and provides the pulsed current to the Schottky diode 16 to drive the tunnel current bias modulation. A pulse inductor 53 provides a low impedance return for the intermediate frequency signal and the direct current bias. Because the inductance of the diode 16 and associated bond wires will decrease the peak pulse voltage to the diode 16, this inductance is preferably reduced by using a pair of bond wires for each electrical connection. The diode 16 must see a short circuit at high frequencies, increasing the harmonic content of the pulsed first control signal. The short circuit is provided by a short-circuit lowpass filter 33, which has a cutoff frequency of half the first sampling frequency. The short-circuit lowpass filter 33 receives the first mixed signal and suppresses the first sampling frequency, preventing overload of the signal processing circuitry. The lowpass filter 33 further provides a low impedance return for the pulse generator 17. In the preferred embodiment, the lowpass filter 33 is a lumped element filter. A capacitor 22 blocks the diode 16 bias voltage while passing the first mixed signal down to the frequency range where the 1/f noise of the diode 16 overtakes the broadband shot noise current. The first mixed signal is then delivered to a baseband AC amplifier 54, which presents a suitable terminating impedance for the short-circuit lowpass filter 33 and sets the baseband noise floor after the diode 16.

The first mixed signal, now a baseband signal, may be routed through a frequency converter 50. This is not a necessary step, but it can provide a more practical realization by allowing a sampling frequency that is much higher than the analog-to-digital converter (“ADC”) 47 sampling rate. Because most signals of interest are undersampled, doubling the sampling frequency will produce about a 3 decibel improvement in signal to noise ratio. Within the frequency converter 50, the first intermediate frequency mixer 40 provides frequency conversion to a first IF by mixing the first mixed signal with a signal generated by the first local oscillator 43. The first local oscillator 43 is preferably a frequency synthesizer that is in phase lock with the master clock oscillator 28 and produces a signal of ¼ the master clock oscillator 28 frequency. Preferably, the first IF is 1005.1 MHz, which is within a suitable range to allow the use of an inexpensive inline surface acoustic wave (“SAW”) filter for the first IF filter 41. The first IF filter 41 then provides image rejection in the down-converted signal to improve the performance of a first interstage amplifier 23, which amplifies the signal before delivering it to the second IF mixer 56. The second IF mixer 56 uses the frequency provided by the frequency multiplier 55 to convert the first mixed signal to a frequency of 10.7 MHz. This allows the use of a ceramic filter as a second IF filter 57, which provides high quality noise filtering of the signal. The resulting signal is amplified at 10.7 MHz by a second interstage amplifier 24. A third sampling mixer 42 mixes the amplified signal with a signal from a frequency divider 27, which receives the master clock oscillator 28 signal and divides by 4 to provide a signal of 11.33 MHz. This mixing converts the first mixed signal down to a suitable range for the ADC 47 sampling rate, which is received from a divider 34 that divides the master clock oscillator 28 frequency by 16. Switches 37 and 38 are used to bypass the frequency converter 50. An anti-alias lowpass filter 39 provides anti-aliasing filtering of the baseband first mixed signal when the frequency converter 50 is bypassed. Because the high frequency rolloff caused by the intrinsic capacitance of the Schottky diode 16 is not a problem when the converter 50 is bypassed, less gain is needed in the system. A threshold amplifier 29 is used to overcome the ADC 47 noise floor for both cases.

With a first control signal emanating from the master clock oscillator 28 at 45.2 MHz, the frequency divider 27 signal is 11.3 MHz, the first sample frequency is 994.4 MHz, the ADC 47 sampling frequency is 2.825 MHz, and the baseband first mixed signal ranges from 0 to 452 MHz. With a second control signal emanating from the master clock oscillator 28 at 46.173 MHz, the frequency divider 27 signal is 11.543 MHz, the first sample frequency is 1015.8 MHz, the ADC 47 sampling frequency is 2.886 MHz, and the baseband first mixed signal ranges from 0 to 461.7 MHz. These frequencies are chosen to allow the use of low cost ceramic and SAW filters. Additionally, a sampling frequency at or near 1 gigahertz allows the use of smaller Fourier transforms during signal processing. The smaller transforms account for both random variation in detected frequencies and frequency drift in either the master clock oscillator 28 or frequency multiplier 55. Noise from the baseband AC amplifier 54 may be filtered by a second lowpass filter 21.

The baseband signal is digitized by the ADC 47. A Fourier transform computer 48 computes a large fast Fourier transform (“FFT”) to detect the desired signals, such as the first beat frequency, within the baseband signal. The detection process is repeated to acquire a second beat frequency, and the computer 48 analyzes the vital field by deriving the input frequency from the first and second beat frequencies. The FFT results are processed and displayed on the screen 49.

Because of the narrow equivalent noise bandwidth of a large FFT, the sensitivity can be very high, typically −140 dBm. Therefore, a large area can be covered by a low power transmitter. To improve frequency measurement accuracy, the frequency converter 50 may receive a continuous wave frequency reference from an antenna 30. The antenna 30 is preferably tuned to the 2.4 GHz frequency range, and the converter 50 operates with the local oscillator now on the low side instead of the high side. The 2.4 GHz signal is typically phase locked to a GPS disciplined OCXO, and the FFT computer can use the measured frequency error to correct timebase errors in real time to typically 0.1 ppb Low noise amplifier 31 and high pass filter 32 improve the receiver sensitivity, and switch 22 provides isolation at the image frequency when the converter is using the low band input.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A device for detecting vital fields, the device comprising:
a) a tunneling barrier that, when vital waves from the vital field are incident on the tunneling barrier, generates a mixed signal from a control signal and a component of the vital waves;
b) a pulse generator in electrical communication with the tunneling barrier and configured to communicate the control signal to the tunneling barrier; and
c) signal processing circuitry configured to receive the mixed signal from the tunneling barrier and determine a beat frequency from the mixed signal.

2. The device of claim 1 wherein the tunneling barrier is incorporated within a Schottky diode.

3. The device of claim 2 further comprising a current source configured to apply a substantially constant direct current to the Schottky diode such that an average tunnel current is maintained through the Schottky diode.

4. The device of claim 3 further comprising control circuitry configured to generate the control signal and impart a sampling frequency on the control signal, wherein the pulse generator communicates the control signal to the Schottky diode by:
a) receiving the control signal from the control circuitry; and
b) delivering current pulses to the Schottky diode at the sampling frequency, such that each current pulse produces a peak tunnel current in the Schottky diode and the beat frequency is generated during the peak tunnel current in the Schottky diode when vital waves are present.

5. The device of claim 2 wherein the Schottky diode comprises first and second electrodes having high sheet resistivity.

6. The device of claim 5 wherein the Schottky diode further comprises:
a) a semiconducting substrate having a substantially flat surface;
b) a conductive first bonding pad disposed on the surface of the substrate and configured to receive one or more bonding wires;
c) a heavily doped region within the substrate and in electrical communication with the first bonding pad;
d) a lightly doped region of the substrate, the lightly doped region forming the first electrode and being in electrical communication with the heavily doped region;
e) a tunnel barrier disposed on the surface of the substrate over the first electrode;
f) a conductive thin film disposed on the tunnel barrier, the thin film forming the second electrode; and
g) a conductive second bonding pad disposed on the surface of the substrate, configured to receive one or more bonding wires, and in electrical communication with the second electrode.

7. The device of claim 6 wherein the tunnel barrier comprises silicon nitride.

8. The device of claim 6 wherein the tunnel barrier is 1.2 nm thick, and wherein the tunnel barrier is deposited on the substrate using PEALD.

9. The device of claim 6 wherein the thin film is 1 nm thick, and wherein the thin film is deposited on the tunnel barrier using PEALD.

10. The device of claim 6 wherein the thin film comprises titanium nitride.

11. The device of claim 6 wherein the Schottky diode further comprises a first oxide trench disposed on the surface of the substrate over one edge of the lightly doped region, and a second oxide trench disposed on the surface of the substrate over the opposite edge of the lightly doped region, the tunnel barrier and thin film extending over the top of each oxide trench to prevent contact with the edges of the lightly doped region.

12. A device for detecting and analyzing vital fields, the device comprising:
a) control circuitry configured to generate a first control signal at a first sampling frequency and a second control signal at a second sampling frequency different from the first sampling frequency;
b) a detector in electrical communication with the control circuitry, the detector comprising:
i. a Schottky diode configured to maintain a tunnel current caused by electron tunneling and having a tunneling region on which vital waves from the vital field are incident;
ii. a pulse generator in electrical communication with the Schottky diode and configured to provide current pulses to the Schottky diode, the current pulses each generating a peak tunnel current in the Schottky diode; and
iii. electromagnetic shielding substantially enclosing the Schottky diode and the pulse generator such that the electromagnetic shielding prevents unwanted electromagnetic interference with the tunnel current but allows the vital waves to pass; and
c) signal processing circuitry in electrical communication with the detector;
wherein:
a) the control circuitry sends the first control signal to the detector;
b) the first control signal causes the pulse generator to send current pulses to the Schottky diode at the first sampling frequency;
c) an input frequency from the vital waves mixes with a harmonic of the first sampling frequency in the tunneling region of the Schottky diode during the peak tunnel current, generating a first mixed signal having a first beat frequency;
d) the Schottky diode sends the first mixed signal to the signal processing circuitry;
e) the control circuitry sends the second control signal to the detector;
f) the second control signal causes the pulse generator to send current pulses to the Schottky diode at the second sampling frequency;
g) the input frequency from the vital waves mixes with a harmonic of the second sampling frequency in the tunneling region of the Schottky diode during the peak tunnel current, generating a second mixed signal having a second beat frequency;
h) the Schottky diode sends the second mixed signal to the signal processing circuitry; and
i) the signal processing circuitry determines the input frequency from the vital waves using the first and second beat frequencies.

13. The device of claim 12 wherein the Schottky diode comprises:
a) a semiconducting substrate having an atomically flat surface;
b) a conductive first bonding pad disposed on the surface of the substrate and configured to receive one or more bonding wires;
c) a heavily doped region within the substrate and in electrical communication with the first bonding pad;
d) a base electrode disposed within the substrate in electrical communication with the heavily doped region;
e) a tunnel barrier disposed on the surface of the substrate over the base electrode, the tunnel barrier comprising a layer of silicon nitride having nano-scale thickness;
f) a top electrode disposed on the tunnel barrier, the top electrode comprising a thin film of titanium nitride; and
g) a conductive second bonding pad disposed on the surface of the substrate, configured to receive one or more bonding wires, and in electrical communication with the top electrode.

14. The device of claim 13 wherein the substrate is silicon, the first and second bonding pads are aluminum, and the base electrode comprises a lightly doped region of the substrate.

15. The device of claim 13 wherein the substrate is gallium arsenide, the first and second bonding pads are gold, and the base electrode comprises a conductor deposited within the substrate by molecular beam epitaxy.

16. The device of claim 13 wherein the detector further comprises a focusing horn connected to the electromagnetic shielding and positioned to concentrate the vital waves into the active region of the avalanche diode.

17. The device of claim 13 wherein the detector further comprises a short-circuit lowpass filter configured to make the first and second mixed signals baseband signals.

18. The device of claim 13 wherein the signal processing circuitry comprises a Fourier transform computer configured to extract frequency data from the first and second mixed signals.

19. The device of claim 19 wherein the signal processing circuitry further comprises a frequency converter configured to simplify the extraction of frequency data by the Fourier transform computer.

20. The device of claim 19 wherein the signal processing circuitry further comprises a screen for displaying frequency data extracted by the Fourier transform computer.

21. A method of detecting and analyzing a vital field, the method comprising:
a) positioning a detector in the vital field so that vital waves from the vital field are incident upon the detector;
b) collecting spectral data of the vital waves from the detector; and
c) comparing the spectral data to records in a reference database to determine if the spectral data matches information gathered on known vital fields.

22. The method of claim 21 wherein collecting the spectral data comprises:
a) generating a first control signal having a first sampling frequency and a second control signal having a second sampling frequency;
b) sending the first control signal to the detector;
c) receiving a first mixed signal having a first beat frequency from the detector;
d) sending the second control signal to the detector;
e) receiving a second mixed signal having a second beat frequency from the detector; and
f) determining the vital waves' input frequency by:
i. finding the beat frequency shift between the first beat frequency and the second beat frequency;
ii. using the beat frequency shift to determine the frequency of a harmonic of the first sampling frequency that mixed with the input frequency; and
iii. adding the first beat frequency to the frequency of the harmonic of the first sampling frequency.

23. The method of claim 22 wherein the detector comprises a Schottky diode with a tunneling region, and wherein positioning the detector in the vital field comprises positioning the Schottky diode so that the vital waves are incident upon the tunneling region.
